(19) United States
(12) Patent Application Publication
(10) Pub. No.: US 2004/0014178 A1
(43) Pub. Date: Jan. 22, 2004

(54) Von willebrand factor-binding proteins from staphylococci

(76) Inventors: Bengt Guss, Uppsala (SE); Lars Frykberg, Storvreta (SE); Martin Nilsson, Uppsala (SE); Karin Jacobsson, Storvreta (SE); Joakim Ahlen, Uppsala (SE)

Correspondence Address: BACON & THOMAS, PLLC, 625 SLATERS LANE, FOURTH FLOOR, ALEXANDRIA, VA, 22314

(21) Appl. No.: 10/381,596

(22) PCT Filed: Apr. 6, 2001
(86) PCT No.: PCT/SE01/00766

(30) Foreign Application Priority Data
Oct. 4, 2000 (SE) 0003573.3

(51) Int. Cl. A61K 39/00; C07H 21/04; C12P 21/04; C12N 1/21; C12N 15/74

(52) U.S. Cl. 435/69.6; 435/252.3; 435/320.1; 514/12; 424/185.1; 536/23.5

(57) ABSTRACT

Von Willebrand factor binding proteins and polypeptides from Staphylococci are disclosed. Further, recombinant DNA molecules coding for said proteins and peptides, plasmids, phages and phagemids comprising the DNA molecules, and microorganisms and microorganisms comprising the recombinant DNA molecules or the plasmids, phages and phagemids are described. Additionally, a method of producing von Willebrand factor binding protein or polypeptide, a method of blocking the adherence of a Staphylococcus to surfaces, immobilized proteins, antigodies, immunogens, purifications methods and determination of the presence of von Willebrand factor in a complex solution, are disclosed.

The invention relates to the field of gene technology and is concerned with recombinant DNA molecules, which contain a nucleotide sequence coding for a protein or polypeptide having von Willebrand-binding activity. Moreover the invention comprises microorganisms (including viruses) containing the aforesaid molecules, and the use thereof in the production of the aforesaid protein or polypeptide and their use in biotechnology.

### BACKGROUND OF THE INVENTION

Staphylococci

Among the coagulase positive staphylococci Staphylococcus aureus is a pathogenic species responsible for a wide variety of diseases in humans like endocarditis, ostemyelitis, sepsis and wound infections (Espersen et al 1999). The largest populations of staphylococci are found in regions of the skin with large numbers of sweat glands and mucous membranes surrounding openings to the body surface.

For a long time the coagulase negative staphylococci (CNS), were considered as non-pathogenic, but during the last two decades they have emerged as the most frequently isolated pathogens in nosocomial infections. This is mainly due to an increased use of biomaterials in human medicine together with a larger population of immuno compromised patients in hospitals and an increased number of antibiotic multiresistant strains. Staphylococcus lugdunensis (Freney et al 1988) is a CNS which belong to the normal skin flora of humans but occasionally this species can cause severe infections like endocarditis, sepicaemia and various deep tissue infections, vascular prosthesis infection, osteomyelitis and skin infections (Espersen et at 1999, Wasserman et al 1999).

The ability of staphylococci to elicit disease in the host is generally due to several virulence factors like expression of adhesins, capsular polysaccharides, toxins and enzymes that can degrade host components combined with the state of the host. Binding of staphylococci to components in plasma and of the extracellular matrix (ECM) at specific sites or structures of the host cells and tissues is thought to be one of the major steps in the initiation of an infection. The binding is dependent upon specific interactions between extracellular proteins of the pathogen and ligands of the host. The relative importance of particular bacterial protein-ligand interactions may vary depending on different factors like the site of infection or the type or stage of the disease. Since many extracellular proteins of pathogenic staphylococci are multifuntional in their binding properties, the role of an individual extracellular protein cannot be judged by considering a selected single binding property. Therefore it is of importance to study these bacterial surface proteins at the molecular level. One aim of the research has been to study the molecular mechanisms of the respective bacterial/host interactions in order to develop new strategies to combat infections caused by staphylococci. The strategy has been cloning and sequencing of the bacterial genes encoding the extracellular proteins interacting with host components and expression of the genes in E. coli to facilitate production of the proteins for further studies. The produced recombinant proteins have been studied with respect to their ability to prevent bacterial infections and their possible use as new biotechnology tools (EP 163 623, EP 294 349, EP 506 923, WO 84/03103).

von Willebrand Factor

von Willebrand factor (vWF) is a large multifunctional glycoprotein, the mature form consisting of 2050 amino acids arranged in four different types of repeats (A through D). vWF is an essential component in the maintenance of hemostasis by supporting platelet adhesion and aggregation to exposed subendothelium in damaged blood vessels, especially under conditions of high shear forces. vWF exists as dimers about 500 kDa in size, or multimers of different sizes up to 20 000 kDa. vWF is synthesised exclusively by endothelial cells and megakaryocytes. The endothelial cells are generating a plasma pool of vWF with a concentration of 5-10 μg/ml as well as an intracellularly stored supply of vWF in Weibel-Palade bodies. Megakaryocytes are responsible for vWF stored within the α-granule of platelets. The largest multimers of vWP, with the greatest thrombogenic potential are present in these different storage compartments, while circulating multimers generally are smaller. vWF mediates platelet adhesion through two distinct platelet receptors, the glycoprotein (GP) Ib in the GP Ib-V-IX complex and the GP IIb-IIIa (also called integrin αIIbβ3). Further, vWF transports and stabilises the coagulation factor VIII. vWF also binds to the endothelial vitronectin receptor (integrin αVβ3) and to various subendothelial components, such as collagens (type I, III and VI), heparin-like glucosaminoglycans, and sulfatides Vischer and de Merloose (1999), Herrmann et al (1997), Ruggeri (1999). Reduced amount of, or malfunctional vWF leads to one of several types and subtypes of von Willebrand disease, which is the most common inherited bleeding disorder (Mohlke et al 1999).

An earlier report by Hartleib et al. 2000 has claimed that Protein A, an IgG-binding protein present on cells of S. aureus is the vWF-binding protein of this species. The present invention does not relate to protein A.

### SUMMARY OF THE INVENTION

The present invention discloses new von Willebrand factor (vWF) binding proteins called, vWb (von Willebrand factor binding protein) from S. aureus and vWbl (von Willebrand factor binding protein from S. lugdunensis) DNA molecules encoding said proteins and applications for their use.

The invention will be described in closer detail in the following, with support of the enclosed examples and drawings.

### DESCRIPTION OF THE DRAWINGS

FIG. 1. Schematic representation of the vWb protein and alignment of inserts from the corresponding gene vWb, isolated from different phagemid clones obtained after panning an S. aureus phage display library against recombinant vWf. S, signal sequence (signal peptidase clevage site is between amino acids 35 and 36 in SEQ ID NO: 3); B, vWf-binding region (amino acids 368-393 in SEQ ID NO: 4). Numbers in brackets indicate how many times an individual clone was found among the 32 clones sequenced.

FIG. 2. Binding studies with phagemid particles displaying the vWF-binding domain. The number of bound phagemid particles is determined as cfu/μl.

FIG. 3. Inhibition study with phagemid particles displaying the vwf-binding domain. The number of bound phagemid particles was determined as cfu ml⁻¹, kcfu (kilo cfu). The phagemid particles were panned against vWf in the presence of antibodies against vWb (circles) or unspecific antibodies (squares) at different concentrations. Values are mean±SD from two experiments.

FIG. 4. Alignment of the 10 repeat units (R1-R10) in region R of vWbl. Since R10 is considerably more diverged than the other repeats, it is separately aligned to more clearly demonstrate the high similarity between the other repeats. Amino acids perfectly conserved in all repeats are indicated with an asterisk and well conserved amino acids between the repeats are indicated with a dot. The numbers indicate the amino acid position in vWbl according to SEQ ID NO: 2

FIG. 5. Schematic presentation of vWbl and alignment of inserts from phagemid clones obtained after pannings against rvWf. The different regions on vWbl are indicated as S (the signal sequence), A (the non repetitive region) and R (encompassing 10 repeated units). The inserts indicated below vWbl (SlvW1-SlvW7) originate from pannings where phagemid particles were eluted by lowering the pH. The insert above vWbl (SlvW8) originates from a panning procedure where phagemid particles were not eluted. Instead E. coli TG1 cells were added directly to the wells and were allowed to get infected. The numbers indicate the positions of amino acids in vWbl as defined in SEQ ID NO: 2.

FIG. 6. Inhibition in binding of phagemid (SlvW5) particles to immobilised rvWf with the recombinant construct vWbl3r. Microtiter wells coated with rvWf were separately incubated with PBS supplemented with vWbl3r or HSA or only with PBS for 1 h. One tenth of the volume (50 μl) was replaced by diluted (50×) phagestock of SlvW5. After incubation for 1 h, the microtiter plates were washed with PBST and subsequently bound phagemid particles were eluted by lowering the pH to 2.1. Aliquots were used to infect E. coli cells and plated on LAA plates. The result is shown as CFU/ml eluate. Each value is the mean of totally four infections from two separate wells and standard deviations are indicated.

### SEQUENCE LISTING

SEQ ID NO: 1. Complete nucleotide sequence of the vwbl gene from S. lugdunensis

SEQ ID NO: 2. The deduced amino acid sequence of the encoded protein vWbl from S. lugdunenis.

SEQ ID NO: 3. Complete nucleotide sequence of the vwb gene from S. aureus.

SEQ ID NO: 4. The deduced amino acid sequence of the encoded protein vWb from S. aureus.

SEQ ID NO: 5. The mapped 24 amino acid sequence of S. lugdunensis that binds vWF.

SEQ ID NO: 6. The mapped 26 amino acid sequence of S. aureus that binds vWF.

SEQ ID NO: 7-16. 67 amino acids long repeat units (R1-10) in the amino acid sequence of S. lugdunensis (SEQ ID NO: 2).

SEQ ID NO: 17. The N-terminal sequence of the purified secreted vWb protein corresponding to amino acids 36-45 in SEQ ID NO: 4.

### DETAILED DESCRIPTION OF THE INVENTION

The present invention relates to recombinant DNA molecules comprising nucleotide sequences, which codes for proteins or polypeptides having vWF-binding activity. The natural sources of these nucleotide sequences are S. aureus strain Newman and S. lugdunensis strain 2342, respectively but with the knowledge of the nucleotide and deduced amino acid sequences presented here, the respective gene or parts of the genes can be isolated from strains of S. aureus and S. lugdunensis, respectively or made synthetically. In particular the knowledge of the deduced amino acid sequence for the part of the respective protein responsible for the vWF-binding activity can be used to produce syntheic polypeptides, which retain or inhibit the vWF-binding. These polypeptides can be labelled with various compounds such as enzymes, fluorescence, luminiscence, biotin (or derivatives of), radioactivity, etc and use e.g. in diagnostic tests such as ELISA- or RIA-techniques.

It is well known in the art that there may be few mismatches of amino acid residues in the amino acid sequence of a protein while the protein still retains its major characteristics. The mismatches may be replaced of one or several amino acids, deletions of amino acid residues or truncations of the protein. Such mismatches occur frequently in genetic variations of native proteins. It is believed that up to 15% of the amino acid residues may be replaced in a protein while the protein still retains its major characteristics. For production of recombinant DNA molecules according to the invention a suitable cloning vehicle or vector, for example a plasmid, phagemid or phage DNA, may be cleaved with the aid of a restriction enzyme whereupon the DNA sequence coding for the desired protein or polypeptide is inserted into the cleavage site to form the recombinant DNA molecule. This general procedure is well known to a skilled person, and various techniques for cleaving and ligating DNA sequences have been described in the literature (e.g. U.S. Pat. No. 4,237,224, Ausubel et al 1991, Sambrook et al 1989). Nevertheless, to the present inventors' knowledge, these techniques have not been used for the present purpose. If the S. aureus strain Newman and/or S. lugdunensis strain 2342, respectively are used as the source of the desired nucleotide sequences it is possible to isolate said sequences and to introduce the respective sequence into a suitable vector in a manner such as described in the experimental part below or, since the nucleotide sequences are presented here, use a polymerase chain reaction (PCR)-technique to obtain the complete or fragments of the vwb and/or wbl genes.

Host that may be used are, microorganisms (which can be made to produce the respective protein or active fragments thereof), which may comprise bacterial hosts such as strains of e.g. Escherichia coli, Bacillus subtilis, Staphylococcus sp. Streptococcus sp., Lactobacilltis sp. and furthermore yeasts and other eukaryotic cells in culture. To obtain maximum expression, regulatory elements such as promoters and ribosome binding sequences may be varied in a manner known per se. The protein or active peptide thereof can be produced intra- or extra-cellular. To obtain good secretion in various systems different signal peptides could be used. To facilitate purification and/or detection the protein or fragment thereof could be fused to an affinity handle and/or enzyme. This can be done on both genetic and protein level. To modify the features of the respective protein or polypeptide thereof the gene or parts of the gene can be modified using e.g. in vitro mutagenesis, or by fusion of other nucleotide sequences that encode polypeptides resulting in a fusion protein with new features.

The invention thus comprises recombinant DNA molecules containing a nucleotide sequence, which encodes for a protein or polypeptide having vWF-binding properties. Furthermore the invention comprises vectors such as e.g. phagemids, plasmids and phages containing such a nucleotide sequence, and organisms, especially bacteria as e.g. strains of E. coli and Staphylococcus sp., into which such a vector has been introduced. Alternatively, such a nucleotide sequence may be integrated into the natural genome of the microorganism.

The application furthermore relates to methods for production of proteins or polypeptides having the vWF-binding activities of protein vWb and Wbl, respectively or fragments thereof. According to this method, a microorganism as set forth above is cultured in a suitable medium, whereupon the resultant product is isolated by some separating method, for example ion exchange chromatography or by means of affinity chromatography with the aid of vWF bound to an insoluble carrier.

The invention also comprises a method to express and display an vWF-binding protein or parts thereof on a suitable virus particle e.g. bacteriophages like M13 or derivatives thereof.

Vectors, especially plasmids, which contains the respective genes vwb or wbl or parts thereof may advantageously be provided with a readily cleavable restriction site by means of which a nucleotide sequence, that codes for another product, can be fused to the respective nucleotide sequence, in order to express a so called fusion protein. The fusion protein may be isolated by a procedure utilising its capacity of binding to vWf, whereupon the other component of the system may if desired be liberated from the fusion protein. This technique has been described at length in WO 84/03103 in respect of the protein A system and is applicable also in the present context in an analogous manner. The fusion strategy may also be used to modify, increase or change the activity of proteins vWb and Wbl, respectively, (or parts thereof) by fusion the proteins together or with other proteins.

The invention can also be used to affinity purify vWF. The respective recombinant rvWF-binding protein or parts thereof can be expressed and purified and the isolated protein or polypeptide can be bound to an insoluble carrier. The immobilized vWF-binding protein can be used to detect and affinity purify vWF from solutions like serum. The present invention also applies to the field of biotechnology that concerns the use of bacterial extracellular components as immunogens for vaccination against staphylococcal infections (EP 163 623, EP 294 349, EP 506 923). Immunisation using whole bacteria will always trigger a highly polyclonal immun response with a low level of antibodies against a given antigenic determinant. It is therefore preferable to use the protein, polypeptide or DNA according to the present invention for immunisation therapies. Notably, immunisation therapies can be conducted as so called passive and active immunisation. Passive immunisation using the invention proteins or DNA involves the raising of antibodies against the said protein or protein encoded by the administrated DNA in a suitable host animal, preferably a mammal, e.g. a healthy blood donor, collecting and administrating said antibodies to a patient. Another way of generating antibodies for passive immunisation could involve production of specific antibodies in cell cultures. One preferred embodiment is passive immunisation of a patient prior to surgery, e.g. operations involving foreign implants in the body. Active immunisation using the inventive protein or DNA involves the administration of the said protein or DNA to a patient, preferably in combination with a pharmaceutically suitable immunostimulating agent. Examples of such agents include, but are not limited to the following; cholera toxin and/or derivatives thereof, heat labile toxins, such as E. coli toxin and similar agents. The composition according to the present invention can further include conventional and pharmaceutical acceptable adjuvant, well known to a person skilled in the art of immunisation therapy. Preferably, in an immunisation therapy using the inventive DNA or fragments thereof, said DNA is preferably administrated intramuscularly, whereby said DNA is incorporated in suitable plasmid carriers. An additional gene or genes encoding a suitable immunostimulating agent can preferably be incorporated in the same plasmid.

Said immunisation therapies are not restricted to the above described routes of administration, but can naturally be adapted to any one of the following routes of administration: oral, nasal, subcutaneous and intramuscular.

One way of treatment of von Willebrand factor disorders is to administer this factor to a patient using e.g. plasma or recombinant technology produced factor (rvWF) for review see Fischer (1999). One application of the disclosed invention is to affinity purify the vWF from a complex solution like serum which facilitates the purifaction of this factor. Furthermore the invention could also be used to determine the concentration of vWF/rvWF in complex solutions like blood and plasma.

In particular the invention is directed to a von Willebrand factor binding protein or polypeptide from Staphylococci, preferably selected from the group consisting of S. aureus and S. lugdunesis.

In an an embodiment the protein or peptide has an amino acid sequence selected from the group consisting of SEQ ID NO: 2, SEQ ID NO: 4 and SEQ ID NOS: 5-17, and antigen determinant comprising parts thereof. The antigen determinant comprising part of one of the disclosed amino acid sequences comprises at least 5, nomally at least 7, e.g at least 9 amino acid residues.

The invention is also directed to a recombinant DNA molecule comprising a nucleotide sequence coding for a protein or polypeptide according to the invention.

In an embodiment the recombinant DNA molecule comprises at least one nucleotide sequence selected from the group consisting of SEQ ID NO: 1, SEQ ID NO: 3, and nucleotide sequences coding for proteins and peptides having amino acid sequences selected from SEQ ID NO: 2, SEQ ID NO: 4 and SEQ ID NOS: 5-17, and antigen determinant comprising parts thereof.

The invention is further directed to a plasmid, phage or phagemid comprising a DNA molecule according to the invention, and to a microorganism comprising at least one recombinant DNA molecule according to the invention, or at least one plasmid, phage or phagemid according to the invention.

An other aspect of the invention is directed to a method for producing a von Wlllebrand factor binding protein or a polypeptide thereof, comprising the steps of

introducing at least one recombinant DNA molecule according to the invention in a microorganism,

culturing said microorganism in a suitable medium, and

isolating the protein thus formed by chromatographic purification.

Other aspects of the invention comprise a method for producing a von Willebrand factor binding protein or polypeptide thereof, comprising the step of expressing at least one recombinant protein according to the invention on a phage particle to produce a phage particle that shows von Willebrand factor binding activity; a method of blocking the adherence of a Staphylococcus to surfaces, comprising addition of a protein according to the invention, or an antibody according to the invention to a medium containing said Staphylococcus, preferably S. lugdunensis and/or S. aureus.

Still other aspects of the invention are directed to immobilized proteins or peptides according to the invention. The proteins or peptides may be coupled to glass or plastic surfaces, peptides, proteins or carbohydrates, such as Sephadex or Dextran; and antigens specifically binding to a protein or peptide according to the invention. These antibodies may be used for detection of staphylococcal infection.

Yet another aspect of the invention are directed to immunogens comprising a protein or peptide according to the invention. These may preferably be used in vaccines.

Further aspects of the invention comprise a method of purifying von Willebrand factor from a complex solution comprising chromatography with the immobilized protein of the invention, and a method of determining the presence of von Willebrand factor in a complex solution comprising the step of using a protein or peptide according to the invention.

### EXAMPLES

Starting Materials

Bacterial Strains, Phases and Cloning Vectors

S. aureus strains used: Newman, 8325-4, Wood 46, Ö 25, L141, U 2, 12, 73. S. lugdunensis strains used: G5-87, G2-89, G16-89, G6-87, G58-88, G66-88, G3A, SÅ, 2342, 49/90, 49/91, A251 were obtained from Åsa Ljungh (Lund, Sweden). E. coli strains used: TG1, DH5-α, BL 21 (DE3), pLysS.

E. coli strain TG1 was used as bacterial host for construction of the library and production of the phage stocks. The E. coli phage R408 (Promega, Madison, Wis., USA) was used as helper phage.

The phagemid vector pG8SAET was used to construct the phagemid libraries (Jacobsson and Frykberg, 1999).

All strans and plasmid or phagemid constructs used in the examples are available at the Department of Microbiology at the Swedish University of Agricultural Sciences, Uppsala, Sweden.

Buffers and Media

E. coli was grown in Luria Bertani broth (LB) or on LA plates (LB containing 1.5% agar) (Sambrook et al 1989) at 37° C. Ampicillin was in appropriate cases added to the E. coli growth media to a final conc. of 50 μg/ml. Staphylococci were grown at 37° C. on bloodagar-plates (containing 5% final conc. bovine blood) or in Tryptone Soya Broth (TSB obtained from Oxoid, Ltd Basingstoke, Hants., England) PBS: 0,05M sodium phosphate pH 7.1, 0.9% NaCl. PBS-T: PBS supplemented with TWEEN 20 to a final conc. of 0.05%.

Preparation of DNA from Staphylococci.

Strains of staphylococci were grown overnight in TSB. Next morning the cells were harvested and the chromosomal DNA prepared.

Proteins and other Reagents

Human fibrinogen was obtained from (IMCO Ltd, Stockholm, Sweden). Human serum albumin (HSA), fibronectin, human IgG and casein were obtained from Signa, St. Louis, USA. Thrombospondin and human vitronectin and human recombinant von Willebrand factor were obtained from Åsa Ljung, Lund, Sweden. DNA probes were labelled with ³²P-ATP by a random-priming method (Multiprime DNA labelling system; Amersham Inc, Amersham, England). Antibodies aginst human vWF was obtained from Kordia, Leiden, Netherlands. Chicken antibodies against recombinant vWb protein were developed by Immunsystem AB, Uppsala, Sweden. Before using the chicken anti-vWb antibodies in various experiments they were affinity purified on a rvWb column. Nitrocellulose (NC)-filters (ECL from Amersham Pharmacia Biotech. alternatively Schleicher&Schüll, Dassel, Germany) were used to bind DNA in hybridization experiments or proteins in Western-blot techniques.

In order to analyse protein samples by native or sodium dodecyl sulphate-polyacrylamid gel electrophoresis (SDS-PAGE), the PHAST-system obtained from Pharmacia LKB Biotechnology, Uppsala, Sweden, was used according to the suppliers recommendations.

Oligonucleotides used were sythesized by Life Technologies AB (Täby, Sweden). Micro Well plates (MaxiSorp, Nunc, Copenhagen, Denmark) were used in panning experiment. Plasmid DNA was prepared using Qiagen Miniprep kit (Qiagen GmbH, Hilden, Germany) and the sequence of the inserts was determined as descibed by Jacobsson and Frykberg (1995, 1998). The sequences obtained were analysed using the PC-gene program (Intelligenetics, Mountain View, Calif., USA). Alternatively, the NTI Vector computer software (Informax Inc., North Bethesda, Md., USA) was used for analysing the sequences obtained.

Routine Methods

Methods used routinely in molecular biology are not described such as restriction of DNA with endonucleases, ligation of DNA fragments, plasmid purification etc since these methods can be found in commonly used manuals (Sambrook et al 1989, Ausubel et al 1991). Ligation reactions were performed using Ready-To-Go T4 DNA Ligase (Pharmacia, Uppsala, Sweden). The PCR reaction was performed on a MiniCycler (MJ Research Inc., Watertown, Mass., USA). DNA sequencing reactions were performed using ThermoSequenase dye terminator cycle sequencing kit (Amersham Pharmacia Biotech) and the samples were analysed using the using the ABI 377 DNA Sequencer (Perlin Elmer, Foster City, Calif., USA) according to the manufacturer's instructions.

### Example 1

Construction of an S. aureus Shotgun Phage Display Library.

The shotgun phage display library was constructed in principal as described by Jacobson and Frykberg (1996, 1998). In short, chromosomal DNA from S. aureus strain Newman was prepared and then fragmented by sonication for different times. Sonicated DNA was analysed on an agarose gel and DNA fragments in the range of 0.5 to 5 kb were made blunt ended by treatment with T4 DNA polymerase. The DNA fragments were then ligated into the pG8SAET phagemid vector using the Ready-To-Go DNA ligase kit (Amersham Pharmacia Biotech). Electroporation of the ligated material into E. coli TG1 cells resulted in 1×10⁷ ampicillin resistant transformants. Part of an overnight culture (4 ml) of the electroporated bacteria was infected with helper phage R408 (10¹² plaque forming units/ml) at a multiplicity of infection of 20 for twenty minutes and mixed with 0.5% soft agar poured onto LA plates supplemented with ampicillin (LAA-plates). After incubation at 37° C. overnight, the phage particles were released from the soft agar by vigorous shaling in LB. The suspension was centrifuged (15,000×g) for 15 minutes, followed by sterile filtration (0.45 μm). The titer of the phage display library was determined to be 1.5×10⁹ colony forming units (cfu)/ml.

### Example 2

Panning of the S. aureus Phase Display Library against vWF.

Microtiter wells (Maxisorp, Nunc, Copenhagen, Denmark) were coated with 10 μl vWF (1 mg/ml) mixed with 190 μl coating buffer (0.05 M NaHCO₃, pH 9.5) and incubated at room temperature (RT), with shaking, for one hour. The wells were then washed three times with phosphate buffered saline, 0.05% Tween 20 (PBS-T). Two hundred microliters of the phagemid library were added to the vWF coated wells, together with casein at a final conc. of 100 μg/ml. Panning was carried out at RT, with shaking, for four hours. After washing extensively with PBS-T, bound phages were eluted with 200 μl of elution buffer (0.05 M NaCitrat, 0.15 M NaCl, pH 2.0) at RT for two minutes. The eluate was neutralised with 25 μl of 2M Tris-buffer, pH 8.7. Different volumes (0.001 to 50 μl) of the eluate was added to 25 μl of stationary phase E. coli TG1 together with LB to adjust the final volume to 200 μl. The infection was allowed to continue for 20-30 minutes before the suspension was spread on LAA-plates, for determining the number of infected bacteria as cfu/μl of eluate. The plates were incubated overnight at 37° C. The colonies were counted and 150 colonies were transferred to two identical replica plates and the rest of the colonies were collected by resuspension in LB-medium at a final volume of 0.5 ml. This suspension was infected with 10 μl helper phage R408 [10¹² plaque forming units (pfu/ml)] for production of enriched phage stocks. The infected bacteria were mixed with 5 ml of 0.5% soft agar, poured on a LAA-plate and incubated at 37° C. overnight. Thereafter, the soft agar were scraped off, 5 ml of LB was added and the mixture vortexed and vigorously shaken for three hours at 37° C. The phagemids were then harvested by centrifugation (15,000×g) for 15 min. and the supernatant were sterile-filtered (0.45 μm). This enriched phage stock were used for subsequent repannings which were carried out as the panning described above, but with the exception that repannings were performed in two hours. The enrichment of clones expressing the E-tag and the increase in cfu from three cycles of panning against vWF are shown in Table 1.

TABLE 1

| Number of panning | cfu/μl | % E-tag positive clones |
|---|---|---|
| 1 | 24 | 8 |
| 2 | 50 000 | 70 |
| 3 | 182 000 | 94 |

### Example 3

Screening and Sequencing of Phagemid Clones Originating from the S. aureus Phase Display Library.

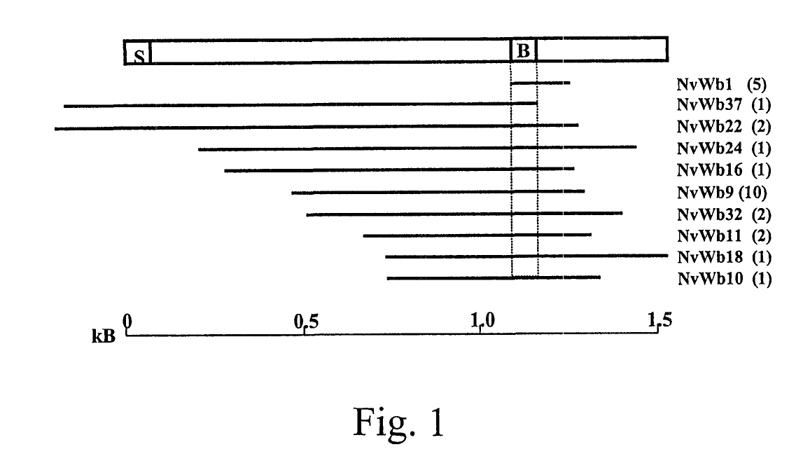

After each round of panning, 150 colonies were picked in identical pattern to two replica-plates, transferred to NC-filters (Schleicher & Schuell, Dassel, Germany) and subsequently screened for expression of the phagemid expression tag (E-tag) with an anti-E-tag antibody (Amersham Pharmacia Biotech). Phagemid DNA from positive clones was prepared and the DNA sequence of the inserts were determined. The obtained sequences were aligned and found to partially overlap each other. Surprisingly, non of the sequenced inserts was homologous to a previous reported S. aureus vWF-binding protein, called protein A (Hartlieb et al 2000). A schematic presentation of the overlapping inserts from different phagemid clones is shown in FIG. 1. Furthermore, the deduced amino acid sequence of the aligned inserts revealed that the binding activity could be mapped to a 26 amino acid long sequence (TSPTTYTETTTQVPMPTVERQTQQQI, SEQ ID NO: 6, corresponding to amino acids 368-393 in SEQ ID NO: 4 and nucleotides 1102-1179, in SEQ ID NO: 3). One phagemid clone, called NvWb32 (in FIG. 1) having an insert with an open reading frame, was chosen for further studies.

### Example 4

Activity of Phagemid Particles of NvWb32.

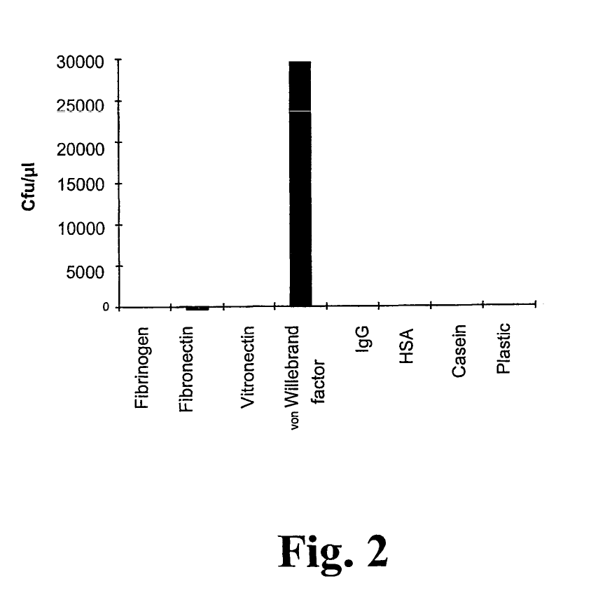

A phagemid stock of NvWb32 was prepared as follows. Five hundred microliters of E. coli TG1 cells harbouring the phagemid were infected with 10 μl helper phage R408 (10¹² pfu/ml). After propagation in soft agar on an LAA plate, the phagemid particles were recovered as described above. The generated phage stock (2×10¹⁰ cfu/ml) was used in an experiment to analyse the binding specificity of the phagemid particles, and it was also used in an inhibition experiment. In the binding specificity experiment, 200 μl of diluted phage stock (1×10⁹ cfu/ml) was panned against untreated microtiter wells (plastic) and microtiter wells coated with 2 μg of either fibrinogen, fibronectin, vitronectin, von Willebrand factor, IgG, HSA or casein. After two hours of panning at RT, the wells were extensively washed with PBS-T and the bound phagemids were eluted and allowed to infect E. coli for determination of cfu/μl of eluate as described above. The results of this experiment are presented in FIG. 2 which clearly shows that NvWb32 has a specificity in binding the vWF.

### Example 5

Inhibition of NvWb32 Binding to vWF Using Antibodies against Recombinant vWb.

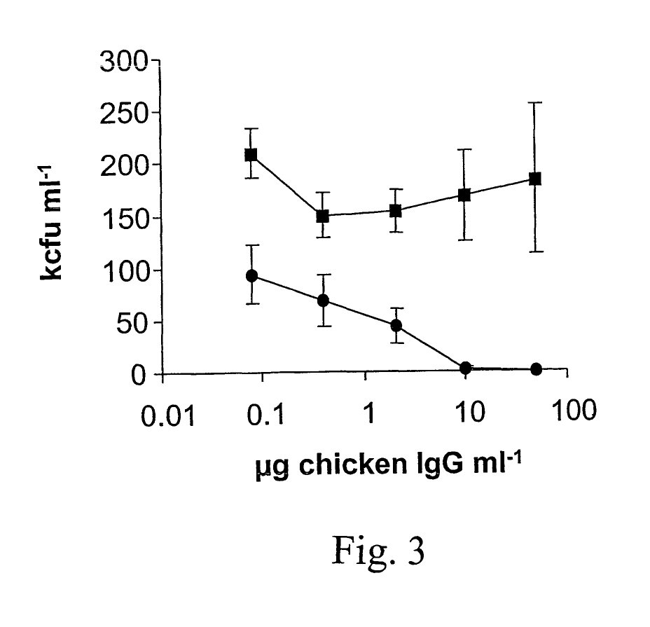

The phage stock NvWb32 was diluted (5×10⁷ cfu/ml) and 90 μl was mixed with 10 μl of various concentrations of chicken antibodies, either unspecific or specific against recombinant vWb (described below). After one hour of incubation at RT, the samples were transferred to vWF-coated microtiter wells (1 μg/well) and incubated further for two hours. The wells were extensively washed with PBS-T and the bound phagemids were eluted and allowed to infect E. coli for determination of cfu/μl of eluate as described above. As seen in FIG. 3 the result of this experiment clearly shows that antibodies raised against recombinant vWb efficiently inhibit the binding of vWb to vWF.

### Example 6

Cloning of the Complete Novel Gene (vwb) Encoding the vWF-Binding Protein from S. aureus.

The genome of S. aureus is public and accessible on DNA data bases like TIGR Microbial Database (http://www.tigr.org/tdb/mdb/mdbinprogress.html). To obtain the complete gene (designated vwb) encoding the vWb protein the DNA inserts of the DNA sequence of the overlapping inserts presented in the example were used to search for homologous sequences in the TIGR S. aureus genome database. Computer search revealed that the overlapping inserts of the clones were contained within an open reading frame of 1551 nt (FIG. 1). Therefore, to isolate the complete vwb gene from S. aureus strain Newman two primers were designed: P1, primers 5′-GAATTCTCATATGATTCATGAAGAAGCC-3′ (downstream) and P2, 5′-GAATTCGCCATGCATTAATTATTTGCC-3′ (upstream) and used in an PCR experiment using Pwo DNA polymerase (Roche Molecular Biochemicals, Mannheim, Germany) with chromosomal DNA from strain Newman as template. The generated PCR product was treated with T4 polynucleotide kinase to generate blunt ends and subsequently ligated into the SmaI-site of the vector pUC18. Part of the ligation was electroporated into E. coli DH5-α for subsequent blue-white screening. Eight white clones were isolated and plasmids were prepared and the respective insert analysed by restriction enzyme analysis, PCR and DNA sequencing. One clone containing the complete gene was further characterized. The nucleotide sequence of the complete vwb gene and the deduced amino acid sequence of the encoded vWb protein are presented in SEQ ID NO: 3 and SEQ ID NO: 4, respectively.

The vwb gene encodes a protein of 517 amino acids with a putative signal sequence but without the cell wall anchoring sequence typical for surface protein in Gram-positive bacteria. This would direct the protein to be exported from of the bacteria and vWb can accordingly be purified from the culture supernatant.

### Example 7

Using Recombinant vWb.

A part of the vwb gene was expressed in E. coli as recombinant vWb (rvWb) using the Impact T7 expression system (New England Biolabs, MA, USA) according to the manufacturer's instructions. The PCR primers P3 (downstream primer: 5′-TTAATACCATGGCTAACCCTGAATTGAAAGACTT-3′) and P4 (upstream primer: 5′-ATTATTATGCGTGTGATTTGAA-3′) were used to amplify the central part of the vwb gene using Taq DNA polymerase from Amersham Pharmacia Biotech. The PCR product was cleaved with NcoI and ligated into pTYB4 vector and subsequently electroporated into E. coli BL 21(DE3) pLys(S). The expressed rvwb was used for generation of antibodies in chicken and for coupling rvwb to HiTrap columns (Amersham Pharmacia Biotech). Using such column, specific anti-vWb antibodies were affinity purified from chicken serum and used in various experiments.

### Example 8

Recombinant vWb can be Used for Purification of vWF from a Complex Solution.

A HiTrap column containing immobilised rvWb was used to affinity purify vWF from human serum. Human serum (15 ml) was passed over the column (which had previously been washed with PBS) the column was thoroughly washed with ten volumes of PBS and five volumes of PBS-T and the bound material was eluted by lowering the pH to 3.0 using 0.1 M Glycin buffer. The eluate was TCA-percipitated as described below. The human vWv was detected in western blots using anti-vWF-antibodies and secondary HRP-labelled antibodies. Bound antibodies were detected with 4-chloro-1-naphtol as substrate. The result clearly showed that recombinant vWb can be used to affinity purify vWF from a complex solution such as serum.

### Example 9

Purification of Wild Type vWb.

S. aureus strain Newman ΔEap, an isogenic mutant strain of S. aureus Newman in which the gene for staphylococcal extracellular adherence protein (Eap) has been deleted, was used for purification of vWb. A culture (containing 100 ml of TSB growth medium) of S. aureus strain ΔEap was harvested in exponential growth phase. After centrifugation the supernatant was sterile filtered and subsequently passed through a HiTrap column with immobilised chicken anti-vWb antibodies. After washing the column with ten volumes of PBS-T and five volumes of PBS the bound material was eluted by lowering the pH to 3.0 using 0.1 M Glycin buffer. The eluate was trichloroacetic acid (TCA)-precipitated as follows: to 1 ml of eluate, 50 μl of 100% TCA was added, the samples were kept on ice for 30 min. and centrifuged in a microcentrifuge for 15 min. at 14.000 rpm at 4° C. The supernatant was discarded and the pellet washed with cold acetone and again centrifuged as above. The supernatant was again discarded, the pellet was dried and resuspended in 10 μl of PBS, pH 7.4. The N-terminal sequence of the purified secreted vWb protein was determined by Edman N-terninal sequencing. The resulting sequence obtained was VVSGEKNPYV (SEQ ID NO: 17) which corresponds to amino acids 36-45 in SEQ ID NO: 2.

### Example 10

SDS-PAGE and Western Blot Analysis of vWb.

Proteins samples were prepared for gel electrophoresis by mixing equal amounts of protein solution with 2×sample buffer (1×sample buffer=62.5 mM Tris-HCl pH 6.8, 10% glycerol, 2% SDS, 5% β-mercapto-ethanol and 0.01% bromophenol blue), boiling the mixture for 5 min. and centrifuging it at 14.000 rpm for 5 min in a microcentrifuge. Supernatants were analysed by SDS-PAGE using the Phast-system (Amersham Pharmacia Biotech) with PhastGel Gradient 8-25% or 4-15% gels and PhastGel SDS Buffer Strips. Proteins were blotted onto nitrocellulose filters by diffision blot. The presence of vWb was detected either with anti-vWb antibodies and secondary RP-labelled antibodies or ¹²⁵I-labelled vWF. The IODO-BEADS lodination Reagent Kit (Pierce, Rockford, Ill., USA) was used to label vWf with ¹²⁵I. Bound antibodies were detected with 4-chloro-1-naphtol and bound ¹²⁵I-labelled vWF was detected with Kodak BioMax MS film (Kodak, Rochester, N.Y., USA). The result clearly shows that vWb can be found in the culture supernatant of S. aureus and that vWF binds to vWb.

### Example 11

The Presence of vwb in Strains of S. aureus.

Chromosomal DNA from different S. aureus strains (83254, Wood 46, Ö 25, L141, U2, 12, 73) was prepared by using the DNeasy Tissue kit from Qiagen. DNA from strain Newman and S. epidermidis strain 19 was also included in the experiment as a positive and negative control, respectively. The DNA was cleaved with EcoRI, separated on a 0.7% agarose gel and blotted to a nitrocellulose filter using the VaccuGene blotting system (Amersham Pharmacia Biotech). After UV-fixation the filter was probed overnight at 65° C. with a ³²P-labelled probe spanning the complet vwb gene. After appropriate washing the filter was put on a Kodak BioMax MR film for 24 hours at −70° C. before developing the film. The result showed that the vwb gene is present in all tested strains of S. aureus.

### Example 12

Construction of Shot-Gun Phase Display Library of Staphyloccus lugdunensis.

A gene library of S. lugdunensis strain 2343 was constructed in principal as described by Jacobson and Frykberg (1996, 1998). In short, chromosomal DNA from strain 2343 was prepared and fragmented by sonication. The sonicated DNA preparation was analysed on an agarose gel and DNA fragments in the range of 0.5 to 5 kb were made blunt ended by treatment with T4 DNA polymerase. The DNA fragments were then ligated into the pG8SAET phagemid vector using the Ready-To-Go DNA ligase kit (Amersham Pharmacia Biotech). Electroporation of the ligated material into E. coli TG1 cells resulted in 2×10⁸ ampicillin resistant transformants. Part of an overnight culture (4 ml) of the electroporated bacteria was infected with helper phage R408 (10¹² plaque forming units/ml) at a multiplicity of infection of 20 for twenty minutes and mixed with 0.5% soft agar poured onto LA plates supplemented with ampicillin (LAA-plates). After incubation at 37° C. overnight, the phage particles were released from the soft agar by vigorous shaking in LB. The suspension was centrifuged (15,000×g) for 15 minutes, followed by sterile filtration (0.45 μm). The titer of the phage display library was determined to be 1×10¹⁰ colony forming units (cfu)/ml.

### Example 13

Panning of the S. lugdunensis Phage Display Library against vWF

A microtiter well (Maxisorp, Nunc, Copenhagen, Denmark) was coated overnight at 4° C. with 200 μl human vWF at a conc. of 25 μg/ml in coating buffer (50 mM NaHCO₃, pH9.7). The well was washed extensively with PBS-T and subsequently blocked for 1 hour at RT with 200 μl of PBS-T supplemented with 1 mg/ml casein. After washing with PBS-T, 200 μl of the phagemid library of S. lugdunensis supplemented with 0.1 mg/ml of casein was added and the well was incubated for 4 hours at RT. Before elution, the well was extensively washed with PBS-T and then eluted with 200 μl buffer solution (50 mM Na-citrate; 150 mM NaCl, pH 2.0). The eluted sample was neutralized by the addition of 25 μl 2M Trs-HCl, pH 8. Afterwards 20 μl of an E. coli TG1 overnight culture was infected with 50 μl of the eluted phage particles supplemented with ˜100 μl of LB broth. After 20 min of incubation at 37° C., the cells were spread on LAA-plates. After incubation overnight at 37° C. the colonies were resuspended in LB broth and pooled. The pooled cells were infected with helper phage, R408 for 20 min at RT and the sample was mixed with 5 ml of LB soft agar (0.5% agar) and poured on a LA plate. After incubation overnight the phagemid particles were extracted and subjected to another round of panning as previous described. The enrichment of clones expressing the E-tag and the increase in cfu from two cycles of panning against vWF are shown in Table 2.

TABLE 2

| Number of panning | cfu/μl | % E-tag positive clones |
|---|---|---|
| 1 | 2 000 | not tested |
| 2 | 80 000 | 95% |

### Example 14

Specificity of the Phagemid Clone SlvW5 Originating from S. lugdunensis Expressing vWF Binding.

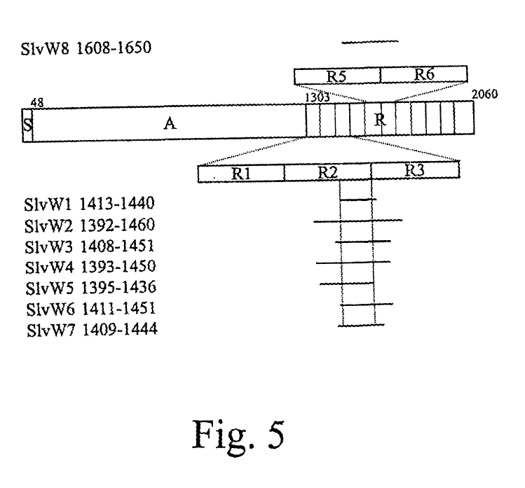

A phage stock of SlvW5 (FIG. 5) was panned against various proteins and plastic. In the binding specificity experiment, 100 μl of phage stock (1.3×10⁹ cfu/ml) was panned against untreated microtiter wells (plastic) and microtiter wells coated with 30 μg/ml of either fibrinogen, fibronectin, vitronectin, von Willebrand factor, IgG, HSA or casein. After two hours of panning at RT, the wells were extensively washed with PBS-T and the bound phagemids were eluted and allowed to infect E. coli for determination of cfu/ml of eluate as described above. The results of this experiment is presented in Table 3 which clearly shows that SlvW5 has a specificity in binding the vWF.

TABLE 3
Results from panning a phage stock (SlvW5)
against immobilised ligands.

| | The number of phagemid |
|---|---|
| Ligands | particles per ml eluates (pH 2.1)^{a,b} |
| rvWf | 3.2 × 10⁸ ± 5.9 × 10⁷ |
| Fibrinogen | 8.6 × 10⁴ ± 1.2 × 10⁴ |
| Fibronectin | 4.6 × 10⁴ ± 2.0 × 10⁴ |
| IgG | 8.0 × 10⁴ ± 2.6 × 10⁴ |
| Vitronectin | 2.4 × 10⁵ ± 3.7 × 10⁴ |
| HSA | 4.1 × 10⁵ ± 4.2 × 10⁴ |
| Thrombospondin | 2.5 × 10⁵ ± 3.5 × 10⁴ |
| —^{c} | 4.0 × 10⁵ ± 3.1 × 10⁴ |

^{a}Determined as colony forming units after infection of E. coli TG1 cells (CFUs) on LA plates supplemented with ampicillin.
^{b}Values are means ± standard deviations (six samples from three separate microtiter wells).
^{c}Uncoated wells.

### Example 15

Screening and Sequencing of Phagemid Clones Originating from the S. lugdunensis Phase Display Library.

A number of the vWF-binding clones (FIG. 5) were chosen for further studies and the DNA sequence of the inserts were determined. Sequence analysis revealed different overlapping insert coding for vWF-binding. Further analysis of the nucleotide sequences showed that all inserts contained an open reading frame (ORF). Computer search using the BLAST (Basic Local Alignment Search Tool) program, where homologies of sequences are analysed revealed that the inserts originating from S. lugdunensis were not homologous to protein A and vWb of S. aureus or to any other sequence in the data base. Furthermore, by comparing the insert of the different clones the vWF-binding activity was mapped to a sequence [FIG. 5, nt 1346-1369 in SEQ ID NO: 1] which corresponds to a 24 amino acid long region [WQYTGQTTTEDGITTHIYQRIQSE, SEQ ID NO: 5].

### Example 16

Cloning and Sequencing of a Gene Encoding a vWf-Binding Protein from S. lugdunensis.

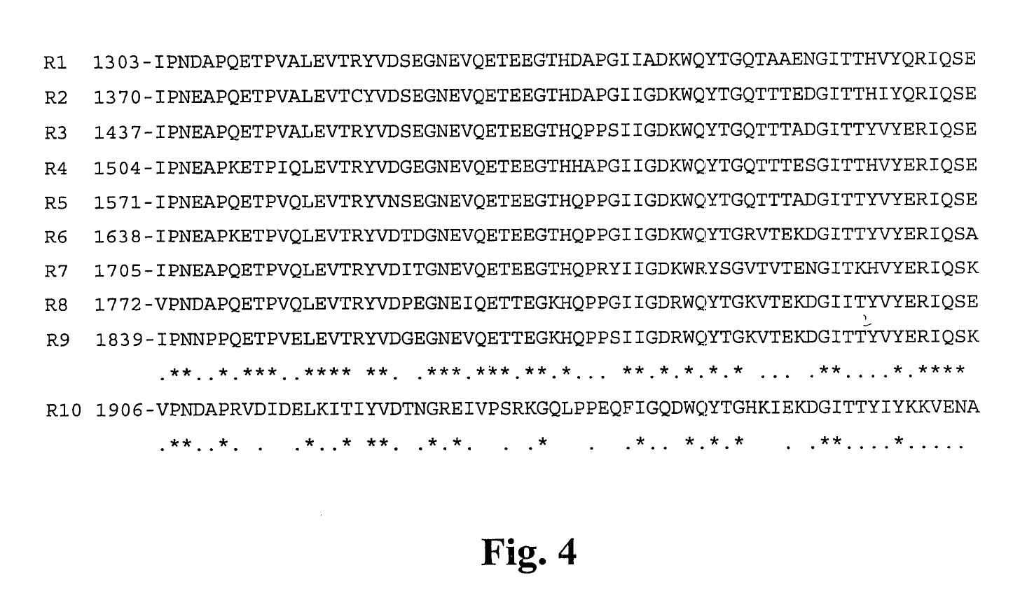

To isolate the complete gene encoding the putative vWf-binding protein, a Southern blot analysis against chromosomal DNA of strain 2342 was performed. The insert of phagemid clone SlvW2 (aa 1392-1460 in SEQ ID NO: 2) was labelled, in a PCR procedure, and used as a probe. An ˜4 kb EcoRI fragment was subsequently ligated into the corresponding site of pUC18. Sequence analysis revealed that the chromosomal fragment contained the 3′-end of the gene but lacked the 5′-end. Thus, to isolate the remaining portion of the gene, an additional Southern blot experiment was conducted, using a probe comprising a fragment from the 5′-end of the EcoRI insert. Based on the results from a Southern blot experiment an ˜3.2 kb HincII fragment was ligated into the SmaI site of pUC18 and subsequently the sequence of the insert was determined. Alignment of the EcoRI fragment and the HincII fragment revealed a putative ORF of 6180 nucleotides starting with a TTG codon (nucleotides 22-24, SEQ ID NO:1). The ORF is preceded by a typical ribosomal binding sequence, situated 10-17 nucleotides upstream the start codon. The gene, termed vwbl, encodes a putative protein of 2060 amino acids, SEQ ID NO: 2, named vWbl (von Willebrand-binding protein of S. lugdunensis). vWbl has a putative signal sequence and the most likely site for cleavage is located between amino acid position 47 and 48 (SEQ ID NO 2). Based on the proposed signal sequence, the mature vWbl consists of 2013 amino acids with a predicted molecular mass of 226 kDa. Following the signal sequence there is a region, termed A, consisting of 1255 amino acids (see FIG. 5). The A-region has no apparent similarity to other proteins but it harbours the interesting motif, Arg-Gly-Asp (RGD), situated at position 1134 to 1136 in vWbl (SEQ ID NO: 2), a motif found in many integrin-binding proteins in mammalians as well as in cell surface proteins of several pathogens. The A-region is followed by a repeat region consisting of ten units, termed R1-R10, where each unit comprises 67 amino acids (SEQ ID NOS: 7-16). An alignment of the ten repeat units shows high similarity between them (FIG. 4). The C-terminal part of vWbl harbours several characteristic features found in cell surface bound proteins of Gram-positive bacteria.

### Example 17

Twenty Four Amino Acids Constitutes the “Minimal” vWf-Binding Region in vWbl.

The vWf binding region was mapped by aligning the different phagemid inserts from the panning experiments. This is schematically illustrated in FIG. 5. Despite the high similarity between most of the repeats (FIG. 4), inserts from three different panning experiments comprised the C-terminal end of the R2 unit (SlvW1-SlvW7 in FIG. 5). Based on the alignment the “minimal” vWf-binding region in vWbl was determined, from phagemid clones SlvW1 and SlvW5, to comprise 24 amino acids ranging from position 1413 to 1436 (SEQ ID NO: 2). However, in an additional panning experiment the panning procedure was changed. Instead of eluting phagemid particles with low pH, E. coli TG1 cells were added directly to the wells and allowed to become infected with bound phagemid particles, followed by spreading the bacteria on LAA-plates. This resulted in isolation of phagemid particles comprising parts of the R5 and R6 units (SlvW8 in FIG. 5) as well as clones containing the R2 unit.

### Example 18

Phagemid Clone SlvW5 Binds Specifically to vWf and the Binding can be Inhibited by Recombinant Protein Comprising Regions R1-R3.

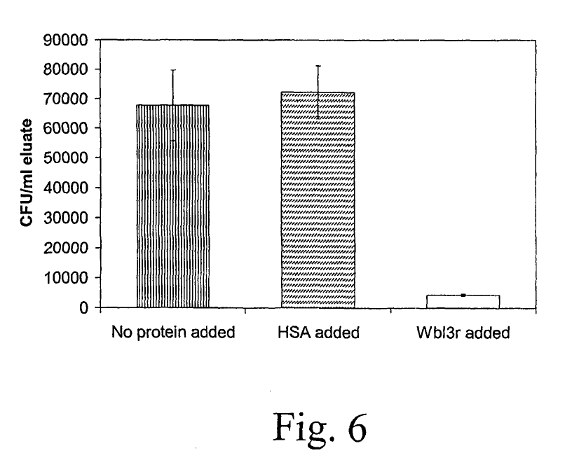

To investigate the binding of vWbl to vWf, a phage stock, derived from SlvW5, was generated. The phage stock was separately panned against seven host proteins and uncoated microtiter wells. The proteins used in the assay were vWf, Fg, fibronectin, IgG, vitronectin, HSA and thrombospondin. Approximately 1000 times more phagemid particles bound to vWf than to the other proteins in the assay (Table 3). In an inhibition assay the same phage stock was used together with purified recombinant protein, termed vWblr3, comprising the C-terminal end of the A-region and repeat units R1-R3 (positions 1247 to 1503 in SEQ ID NO: 2). vWblr3 was incubated in vWf coated microtiter wells prior to the addition of the phage stock. As shown in FIG. 6 the phage binding was inhibited approximately 95% compared to the controls.

### Example 19

Clinical Isolates of S. lugdunensis Possess vwbl or vwbl Like Genes.

To investigate the distribution of the vwbl gene among clinical isolates of S. lugdunensis chromosomal DNA was purified from strain 2342 and 11 other strains (G5-87, G2-89, G16-89, G6-87, G58-88, G66-88, G3A, SÅ, 49/90, 49/91 and A251) of this species, and used in Southern blot analysis. The DNA preparations were digested with EcoRI and probed in the Southern blot with purified PCR product covering units R1-R10. All strains were found to possess a fragment that reacted with this probe. In addition, we performed PCR, with primers based on the sequence just upstream and downstream of the repeat region, in the respective DNA samples. A fragment was amplified from ten of the twelve strains. Interestingly, the size of the PCR products varied, indicating that the number of repeat units in vwbl differs between S. lugdunensis strains. It was then possible to divide the ten strains into four groups, according to the sizes of the generated PCR fragments.

### REFERENCES

Ausubel, F. A., Brent, R., Kingston, R. E., Moore, D. D., Seidman, J. G., Smith, J. A. and Struhl, K.(eds) (1991) Current Protocols in Molecular Biology, Greene Publishing and Wiley-Intersciences, New York.

Espersen, F., Hedström, S. Å. and Solberg, C. O. (Editors) The ever present pathogens (999), ISBN Published by Rosell & Co , Göteborg, Sweden.

Fisher, E. B. (1999) Rekombinant von Willebrand Factor: Potential Therapeutic Use. J. Thrombosis and Thrombolysis 8:197-205.

Freney, J., Brun, Y., Bes, M., Meugnier, H., Grimont, P. A. D., Nervi, C. and Fleurette, J. (1988). Staphylococcus lugdunensis sp. nov. and Staphylococcus schleiferi sp. nov., two species from human clinical specimens. Int. J. Syst. Bacteriol. 38:168-172.

Hartleib, J., Köhler, N., Dickinson, R. B., Chhatwal, G. S., Sixma, J. J., Hartford, O. M., Foster, T. J., Peters, G., Kehrel, B. E. and Herrmann, M. (2000) Blood 96:2149-2156.

Herrmann, M., Hartleib, J., Kehrel, B. E, Montgomery, R., Sixma, J. J. and Peters, G. (1997) Interaction of von Willebrand factor with Staphylococcus aureus. J. Infect. Dis. 176:984-991.

Jacobsson, K. and Frykberg, L. (1996) Phage display shot-gun cloning of ligand-binding domains of prokaryotic receptors approaches 100% correct clones. BioTechniques 20:1070-1081.

Jacobsson, K. and Frykberg, L. (1998) Gene VIII-based, phage-display vectors for selection against complex mixtures of ligands. BioTechniques 24:294-301.

Jacobsson and Frykberg (1999) pp237-238 in Expression genetics: Accelerated and high throughput methods. Eaton Publishing. Edited by McClelland, M. and Pardee, A. B. ISBN 1-881299-24-4

Li, D.-Q., Lundberg, F. and Ljung, Å. (1999) Binding of von Willebrand factor by coagulase-negative staphylococci. J. Med. Microbiol. 49:1-9.

Mohlke, K. L., Nichols, W. C. and Ginsburg, D. (1999) Int. J. Clin. Lab. Res. 29:1-7.

Navarre, W. and Schneewind, O. O. Surface proteins of gram-positive bacteria and mechanisms of their targeting to the cell wall envelope. Microbiology and Molecular Biology Reviews. 63:174-229.

Paulsson, M., Petersson, A. and Ljungh, Å. (1993) Serum and tissue protein binding and cell surface properties of Staphylococcus lugdunensis. Med Microbiol. 38:96-102.

Ruggeri, Z. M. (1999) Structure and function of von Willebrand factor. Trombosis and Haemostasis 82:576-584.

Sambrook, J., Fritsh, E. F. and Maniatis, T. (1 989) Molecular cloning, A laboratory manual, second ed. Cold Spring Harbour Laboratory Press, New York.

Vischer, U. M. and de Moerloose, P. (1999) von Willebrand factor: from cell biology to the clinical management of von Willebrand's disease. Critical Reviews in Oncology Hematology 30:93-109.

Wasserman, E., Lombard, L. and Walzi, G. (1999) Staphylococcus lugdunensis endocarditis in a young, previously healty female. Eur. J. Clin. Microbiol. Infect. Dis. 18:289-291.

Patents or Patent Applications Cited:

EP 163 623

EP 294 349

EP 506 923

U.S. Pat. No. 4,237,224

WO 84/03103

1. von Willebrand factor binding protein or polypeptide from Staphylococci having an amino acid sequence selected from the group consisting of SEQ ID NO: 2, SEQ ID NO: 4 and SEQ ID NOS: 5-17, and antigen determinant comprising parts thereof.

2. Recombinant DNA molecule comprising a nucleotide sequence coding for a protein or polypeptide according to claim 1.

3. Recombinant DNA molecule according to claim 2, comprising at least one nucleotide sequence selected from the group consisting of SEQ ID NO: 1, SEQ ID NO: 3, and nucleotide sequences coding for proteins and peptides having amino acid sequences selected from SEQ ID NO: 2, SEQ ID NO: 4 and SEQ ID NOS: 5-17, and antigen determinant comprising parts thereof.

4. Plasmid, phage or phagemid comprising a DNA molecule according to claim 2 or 3.

5. Microorganism comprising at least one recombinant DNA molecule according to claim 2 or 3, or at least one plasmid, phage or phagemid according to claim 4.

6. Method for producing a von Willebrand factor binding protein or a polypeptide thereof, comprising the steps of
introducing at least one recombinant DNA molecule according to claim 2 or 3 in a microorganism,
culturing said microorganism in a suitable medium, and
isolating the protein thus formed by chromatographic purification.

7. Method for producing a von Willebrand factor binding protein or polypeptide thereof, comprising the step of
expressing at least one recombinant protein according to claim 1 on a phage particle to produce a phage particle that shows von Willebrand factor binding activity.

8. Method of blocking the adherence of a Staphylococcus to surfaces, comprising addition of a protein according to claim 1, or an antibody according to claim 11, to a medium containing said Staphylococcus.

9. Method according to claim 10, wherein the Staphylococcus is selected from S. lugdunensis and S. aureus.

10. Immobilized protein or peptide according to claim 1.

11. Antibodies specifically binding to a protein or peptide according to claim 1.

12. Immunogen comprising a protein or peptide according to claim 1 or 10.

13. Method of purifying von Willebrand factor from a complex solution comprising chromatography with the immobilized protein of claim 10.

14. Method of determining the presence of von Willebrand factor in a complex solution comprising the step of using a protein or peptide according to claim 1 or 10.


SEQUENCE LISTING (17 sequences)

SEQ ID NO: 1 - DNA, 6204 bp, Staphylococcus lugdunensis
caattaaggagagagaacccattgaaaaggaagaaaagcaaacaaaaagattatttctcgaatgttcaaaataaatactccataagaaaattttcagtaggaattacatcaattttaataggagcatctgtcatttttggagctaattctgaagcacatgctgcagagataaaaagtgatgataatacacaaaaagttgttaataaagaatatagcgatggtatttcgcaagaacaacaggataaatcattaaatcttgctcaagacaaagaaaagtcaaataatgttaataaaaataaagtaacagacgtaggaacatctgatgtgttaaaggatacaaagacagcacatgaaaatgtacagcaacaagataatgttgcttcaaaagaagaaactactaaaaaaacgcctagtgctatagataaaaataatacatcagcacaatctgaagttgtaacttcgagtgatgctactaagacatctactgctttaaatcaagttgatagaaatcaactatcatcacaatctacttcagaaagaccgaaaaaacgcgttaaacgtgacgtgggtactgatgacaagcaattagtaggggaattcgtatttagcggcacaaatagtaatgaaagacgctataatttaacatcagatatcgtgtctaacagacgtgtaacaagtacgtcattttcttattcagcatcagggcatggcacaattgataatggcaagctggtttacgaagcacctaaaaaatttataatttcagaaccaacattttcaaaatcggaatttgtgactaacaagacaaatttatctgataatgagacatggcgctatcaatttgatttaagaccactttctggtacatcagcaggtcaaatcaatatcagtcaagttattggcgggatgatttggacgggaccaggtgaaggtgatgttgttacttcaacaatgaaaatgtatcagggagatacgcttgttgatactaaacaagttaatgcaacatttgatcatataaaaggtggtttatacatagaccgagggcaagaaaaaccgacgtctatttggagaattacacgaaaatcagcggctgcacaatcacctacgattggagttatcaaagaagatgggacaatttctgagaaaacggataactatcgctatcaggtgccactgccaagagataattggtttttagaaaatcgggatgatgcagtatcaccacattattattcacgttatactgattttaaatataatattttaaatattccaagttggttagaacttgatccagatgctaaaggcaatagattttggacacaagatgaatcaggcattcatctccatcttaatgaaggtgatattttaccttataatggttatacacccgtattaaaattgaaaaaatctgcattaacaccagaattattagcaaaatttaaaactgacggctttattaaagtaaacttagattggcaaacgataggccgacttccaaatggacaagattatattcaaaatgtacctgatccagagggaattaagtttaaaatgattaatgaggctggtacagcaacaagtaatgtttattttatgaatttccgactgcaagagccttcacatttgttttcaaaagcagatcacaaaaatgaagtattccgtgtgcaaaaatatatatatgatagagaccaatcgaataaatatgcacctacttacatgcattcaatacaattaccatcagggcaagaaggcgattactttactacctttaagctttcattacctagaataaattataataacggttcaagtgataaaaatgcagataaaggaataatattaaaagcaccatttattttatacggtgtaaatgctgatggatctactacaaaattaaacgaatggacgtcaatcaacaatacaaataatacctatggattgggagataaaaaatataatcatttaatactccaaacaccaattattagagatacggtttcaccagatagtgaagcagaaaaggacatttatggttgggaggcagatatcacatatgcagtagatgatcagcgttgggaaacagctgcaaaagatgatgatgtgacccaaatgcaaaatggaattattattaatcaagtagcagaaaatacgttacaagctgctagttccgttccaacaagggcattgacaggtaatgagttacaaccagcatattcgtatattcatactaaggatgcatttaaacacactttaacagatgttgatgtaagaaatacaaattcaggtttaggaaatgtattaaactataatgaaaaagctaatgtcattgtcaaagcagatactaaagattatatgaaatatttaagtacggatccattagataatagttctgatattagcgttgataaattagatcatattaatcatgtctatttatcattaagtgcgcctgatgacacggcaataggtaatgtgcgtgcatggacgaataatgaattaatacgtacatcaacagtttggaatccttcattatatgaaactacgccatcgaatgttctaaaaacccgccctgtcttaaatccaattaaagtgataaaaaattataaaaatagcggtagaacgttatatatttatgaagcacctgaaggatataagtggaatcagtcggtaaagaattccattacagaacgaagttcaatcactcagaatacgccagaaatcacttttgatatttataatagaggtacgttaccaacgggaacttattcaattagatatgctacgatatgggatgaaaattcggaaattgtgcgtcctactgaagaacaatctttaagtcataataatcttgaattatcttatgtaattacagaagatttaagtggtaataaaaagtttgtctcagttattgatgtgccatttaaaattgcattagctaaggaatatgcttctacattaactattggtaaagatgcgtcgaacagttttgataaatctcaggttgatgttaacttaggagaaagtgtgaatttacaaacaaatacggccaactttactaacagtgaaggaattattaaagaaatcattgtgaccattccgaaagacaatattaaaacgaatttaacggcgttaattcctgacactgaaaaatatcgtgttgtttatacaacagacacggatgtacgtaatggcgtgtacaattctaatccaacagatttaacaaaagttacggcagttaaatatgtttttgatgaaccccttgttttaacaaatggacaaagtttccaaacaaatatgcgtgttactgtgccagaggatgcacctattttaactaaagcgcattctcaaatctttactaaaggtttggataatacatggctttcagggaataaagttgagcttgaaacagaagataaccgtggagacttagtggttaagtatactaatgaatcagggaatacaattcaaaattcactgacatcaaaaggtaaaaagaatacggagtataatgttagtgtgcctcaaatgattgatagactaaatcgacactataaatttgttagggttgataatcaacttgatcctacaacgggtcattatgctaaaggtcaaactaaaattgttaatttaatatacgtagaagtatttgaaggtagtgtgatagccgactataaaacaacggatggagaagtgttaagtccgctagtaacagttgtaaatagtcaaattgaaggaacagaatatacagctacaccagcaacaattccagatcgcgtaacctttgaaacaactgatgacggtaaagttaaaaagacaataagttatcatttaatttcgacaccagaaaatcaatctggaacagttgtaggtaagcagacaatagaagttcactatgtatacgaaccgattacaacttatgaacagataccgaacgacgcgccgcaagaaacgccagttgcgttagaagtaacacgttacgtggatagtgaaggtaatgaagtgcaggaaacggaagagggcacacatgacgcaccaggtattatcgcggataaatggcaatatacaggccaaacagcagcagaaaatggtattacaacacatgtatatcaacgtatccagtcagaaataccgaatgaagcaccacaagagacgccagtcgcgttagaagtaacatgttatgtggatagcgaaggtaatgaagtgcaggaaacggaagagggcacgcatgacgcaccaggtattatcggagataaatggcaatatacaggccaaacaacaacagaagacggtatcacaacgcatatatatcaacgtattcaatcagaaataccgaatgaagcaccacaagagacgccagtcgcgttagaagtaacacgttatgtggatagtgaaggtaatgaggtgcaagagacagaagaaggcacgcatcaaccaccaagtattatcggagacaaatggcaatatacaggtcaaacaacaacggcagatggcatcacaacatatgtgtatgaacgtatccagtcagaaataccaaatgaagcgccgaaggaaacgccaatacaattagaagtaacacgttatgtagatggcgaaggtaatgaagtgcaagaaacggaagaaggcacacatcatgcgccaggtattatcggagacaaatggcaatatacgggtcaaacaacaacagaatctggcatcacgacgcatgtgtatgaacgtatacaatcggaaataccaaacgaagcaccgcaagagacaccggtacaattagaagtaacacgttatgtgaatagtgaaggtaatgaggtgcaagagacagaagaaggcacgcatcaaccaccaggtattatcggagacaaatggcaatatacaggccaaacaacaacagcagatggtatcacaacatatgtgtatgaacgcattcaatcagaaataccaaatgaagcaccgaaggaaacaccggtgcaattagaagtgacacgctatgtggatactgatggaaatgaagttcaagagacagaagaaggcacgcaccaaccgcctggcattattggagataaatggcagtatacaggaagagtcacagaaaaagatggcatcacaacgtatgtatatgaacgcatccaatcagcaatcccgaacgaagcaccgcaagagacaccggtacaattagaagtaacgcgttatgtggatattaccggaaatgaagttcaagagacagaagaaggtacgcatcaaccgcgttatatcattggagataaatggcgttattctggagtaacagtgacagaaaatggtattactaaacatgtctatgaacgcattcaatcaaaagttccaaatgacgcaccacaagaaacgccagtacaattagaagtaacacgctatgtggacccagaaggaaacgaaatacaagaaacaacagaaggtaaacatcaaccgcctggcattattggtgacagatggcaatatacaggaaaagtcacagaaaaagatggcatcataacatatgtttatgaacgtattcagtcagaaataccaaataatccaccgcaagagacaccggtagaattagaagtaacacgctatgtagatggcgaaggtaatgaagtgcaagaaacaacagaaggtaaacatcaaccgcctagcattattggagatagatggcaatacacaggaaaagttacagaaaaagacggcattacaacatatgtctatgaacgtattcaatcaaaagttccaaatgacgcaccgcgtgtagacattgatgaattgaaaatcacaatttatgttgatacaaatggtcgtgaaattgttccatcacgaaaaggtcagttaccaccagaacaatttatcggacaagattggcaatatacaggacataagattgaaaaagatggtattacaacatatatttataaaaaagtagagaatgctgtgccagcaaaacaattgaaaaagactaagcataatacgcagtctgaaagtcaattcaaacatacaccacaagttaaacaacaacttgttaaatatcataatgttaaagaacaacgttctattgaaaagtcagaacatacagatatgcatgtgtcagagttacctgaaacaggagaaacagctaataaaaacggactaataggtggattgttaatagcaataggtgcatttttcgtaacaaaaagaaaaaaagaaaacacaaaataa

SEQ ID NO: 2 - protein, 2060 aa, Staphylococcus lugdunensis
LKRKKSKQKDYFSNVQNKYSIRKFSVGITSILIGASVIFGANSEAHAAEIKSDDNTQKVVNKEYSDGISQEQQDKSLNLAQDKEKSNNVNKNKVTDVGTSDVLKDTKTAHENVQQQDNVASKEETTKKTPSAIDKNNTSAQSEVVTSSDATKTSTALNQVDRNQLSSQSTSERPKKRVKRDVGTDDKQLVGEFVFSGTNSNERRYNLTSDIVSNRRVTSTSFSYSASGHGTIDNGKLVYEAPKKFIISEPTFSKSEFVTNKTNLSDNETWRYQFDLRPLSGTSAGQINISQVIGGMIWTGPGEGDVVTSTMKMYQGDTLVDTKQVNATFDHIKGGLYIDRGQEKPTSIWRITRKSAAAQSPTIGVIKEDGTISEKTDNYRYQVPLPRDNWFLENRDDAVSPHYYSRYTDFKYNILNIPSWLELDPDAKGNRFWTQDESGIHLHLNEGDILPYNGYTPVLKLKKSALTPELLAKFKTDGFIKVNLDWQTIGRLPNGQDYIQNVPDPEGIKFKMINEAGTATSNVYFMNFRLQEPSHLFSKADHKNEVFRVQKYIYDRDQSNKYAPTYMHSIQLPSGQEGDYFTTFKLSLPRINYNNGSSDKNADKGIILKAPFILYGVNADGSTTKLNEWTSINNTNNTYGLGDKKYNHLILQTPIIRDTVSPDSEAEKDIYGWEADITYAVDDQRWETAAKDDDVTQMQNGIIINQVAENTLQAASSVPTRALTGNELQPAYSYIHTKDAFKHTLTDVDVRNTNSGLGNVLNYNEKANVIVKADTKDYMKYLSTDPLDNSSDISVDKLDHINHVYLSLSAPDDTAIGNVRAWTNNELIRTSTVWNPSLYETTPSNVLKTRPVLNPIKVIKNYKNSGRTLYIYEAPEGYKWNQSVKNSITERSSITQNTPEITFDIYNRGTLPTGTYSIRYATIWDENSEIVRPTEEQSLSHNNLELSYVITEDLSGNKKFVSVIDVPFKIALAKEYASTLTIGKDASNSFDKSQVDVNLGESVNLQTNTANFTNSEGIIKEIIVTIPKDNIKTNLTALIPDTEKYRVVYTTDTDVRNGVYNSNPTDLTKVTAVKYVFDEPLVLTNGQSFQTNMRVTVPEDAPILTKAHSQIFTKGLDNTWLSGNKVELETEDNRGDLVVKYTNESGNTIQNSLTSKGKKNTEYNVSVPQMIDRLNRHYKFVRVDNQLDPTTGHYAKGQTKIVNLIYVEVFEGSVIADYKTTDGEVLSPLVTVVNSQIEGTEYTATPATIPDRVTFETTDDGKVKKTISYHLISTPENQSGTVVGKQTIEVHYVYEPITTYEQIPNDAPQETPVALEVTRYVDSEGNEVQETEEGTHDAPGIIADKWQYTGQTAAENGITTHVYQRIQSEIPNEAPQETPVALEVTCYVDSEGNEVQETEEGTHDAPGIIGDKWQYTGQTTTEDGITTHIYQRIQSEIPNEAPQETPVALEVTRYVDSEGNEVQETEEGTHQPPSIIGDKWQYTGQTTTADGITTYVYERIQSEIPNEAPKETPIQLEVTRYVDGEGNEVQETEEGTHHAPGIIGDKWQYTGQTTTESGITTHVYERIQSEIPNEAPQETPVQLEVTRYVNSEGNEVQETEEGTHQPPGIIGDKWQYTGQTTTADGITTYVYERIQSEIPNEAPKETPVQLEVTRYVDTDGNEVQETEEGTHQPPGIIGDKWQYTGRVTEKDGITTYVYERIQSAIPNEAPQETPVQLEVTRYVDITGNEVQETEEGTHQPRYIIGDKWRYSGVTVTENGITKHVYERIQSKVPNDAPQETPVQLEVTRYVDPEGNEIQETTEGKHQPPGIIGDRWQYTGKVTEKDGIITYVYERIQSEIPNNPPQETPVELEVTRYVDGEGNEVQETTEGKHQPPSIIGDRWQYTGKVTEKDGITTYVYERIQSKVPNDAPRVDIDELKITIYVDTNGREIVPSRKGQLPPEQFIGQDWQYTGHKIEKDGITTYIYKKVENAVPAKQLKKTKHNTQSESQFKHTPQVKQQLVKYHNVKEQRSIEKSEHTDMHVSELPETGETANKNGLIGGLLIAIGAFFVTKRKKENTK

SEQ ID NO: 3 - DNA, 1524 bp, Staphylococcus aureus
ttgaaaaataaattgctagttttatcattgggagcattatgtgtatcacaaatttgggaaagtaatcgtgcgagtgcagtggtttctggggagaagaatccatatgtatctgagtcgttgaaactgactaataataaaaataaatctagaacagtagaagagtataagaaaagcttggatgatttaatatggtcctttccaaacttagataatgaaagatttgataatcctgaatataaagaagctatgaaaaaatatcaacagagatttatggctgaagatgaggctttgaagaaattttttagtgaagagaaaaaaataaaaaatggaaatactgataatttagattatctaggattatctcatgaaagatatgaaagtgtatttaatactttgaaaaaacaaagtgaggagttcttaaaagaaattgaagatataaaaaaagataaccctgaattgaaagactttaatgaagaggagcaattaaagtgcgacttagaattaaacaaattagaaaatcagatattaatgttaggtaaaacattttatcaaaactatagagatgatgttgaaagtttatatagtaagttagatttaattatgggatataaagatgaagaaagagcaaataaaaaagcagttaacaaaaggatgttagaaaataaaaaagaagacttagaaaccataattgatgaattttttagtgatatagataaaacaagacctaataatattcctgttttagaagatgaaaaacaagaagagaaaaatcataaaaatatggctcaattaaaatctgacactgaagcagcaaaaagtgatgaatcaaaaagaagcaagagaagtaaaagaagtttaaatactcaaaatcacaaacctgcatctcaagaagtttctgaacaacaaaaagctgaatatgataaaagagcagaagaaagaaaagcgagatttttggataatcaaaaaattaagaaaacacctgtagtgtcattagaatatgattttgagcataaacaacgtattgacaacgaaaacgacaagaaacttgtggtttctgcaccaacaaagaaaccaacatcaccgactacatatactgaaacaacgacacaggtaccaatgcctacagttgagcgtcaaactcagcaacaaattatttataatgcaccaaaacaattggctggattaaatggtgaaagtcatgatttcacaacaacgcatcaatcaccaacaacttcaaatcacacgcataataatgttgttgaatttgaagaaacgtctgctttacctggtagaaaatcaggatcactggttggtataagtcaaattgattcttctcatctaactgaacgtgagaagcgtgtaattaagcgtgaacacgttagagaagctcaaaagttagttgataattataaagatacacatagttataaagaccgaataaatgcacaacaaaaagtaaatactttaagtgaaggtcatcaaaaacgttttaataaacaaatcaataaagtatataatggcaaa

SEQ ID NO: 4 - protein, 517 aa, Staphylococcus aureus
LGKIKEKIQLKNKLLVLSLGALCVSQIWESNRASAVVSGEKNPYVSESLKLTNNKNKSRTVEEYKKSLDDLIWSFPNLDNERFDNPEYKEAMKKYQQRFMAEDEALKKFFSEEKKIKNGNTDNLDYLGLSHERYESVFNTLKKQSEEFLKEIEDIKKDNPELKDFNEEEQLKCDLELNKLENQILMLGKTFYQNYRDDVESLYSKLDLIMGYKDEERANKKAVNKRMLENKKEDLETIIDEFFSDIDKTRPNNIPVLEDEKQEEKNHKNMAQLKSDTEAAKSDESKRSKRSKRSLNTQNHKPASQEVSEQQKAEYDKRAEERKARFLDNQKIKKTPVVSLEYDFEHKQRIDNENDKKLVVSAPTKKPTSPTTYTETTTQVPMPTVERQTQQQIIYNAPKQLAGLNGESHDFTTTHQSPTTSNHTHNNVVEFEETSALPGRKSGSLVGISQIDSSHLTEREKRVIKREHVREAQKLVDNYKDTHSYKDRINAQQKVNTLSEGHQKRFNKQINKVYNGK

SEQ ID NO: 5 - protein, 24 aa, Staphylococcus lugdunensis
WQYTGQTTTEDGITTHIYQRIQSE

SEQ ID NO: 6 - protein, 26 aa, Staphylococcus aureus
TSPTTYTETTTQVPMPTVERQTQQQI

SEQ ID NO: 7 - protein, 67 aa, Staphylococcus lugdunensis
IPNDAPQETPVALEVTRYVDSEGNEVQETEEGTHDAPGIIADKWQYTGQTAAENGITTHVYQRIQSE

SEQ ID NO: 8 - protein, 67 aa, Staphylococcus lugdunensis; identical to SEQ ID NO: 7

SEQ ID NO: 9 - protein, 67 aa, Staphylococcus lugdunensis; identical to SEQ ID NO: 7

SEQ ID NO: 10 - protein, 67 aa, Staphylococcus lugdunensis
IPNEAPKETPIQLEVTRYVDGEGNEVQETEEGTHHAPGIIGDKWQYTGQTTTESGITTHVYERIQSE

SEQ ID NO: 11 - protein, 67 aa, Staphylococcus lugdunensis
IPNEAPQETPVQLEVTRYVNSEGNEVQETEEGTHQPPGIIGDKWQYTGQTTTADGITTYVYERIQSE

SEQ ID NO: 12 - protein, 67 aa, Staphylococcus lugdunensis
IPNEAPKETPVQLEVTRYVDTDGNEVQETEEGTHQPPGIIGDKWQYTGRVTEKDGITTYVYERIQSA

SEQ ID NO: 13 - protein, 67 aa, Staphylococcus lugdunensis
IPNEAPQETPVQLEVTRYVDITGNEVQETEEGTHQPRYIIGDKWRYSGVTVTENGITKHVYERIQSK

SEQ ID NO: 14 - protein, 67 aa, Staphylococcus lugdunensis
VPNDAPQETPVQLEVTRYVDPEGNEIQETTEGKHQPPGIIGDRWQYTGKVTEKDGIITYVYERIQSE

SEQ ID NO: 15 - protein, 67 aa, Staphylococcus lugdunensis
IPNNPPQETPVELEVTRYVDGEGNEVQETTEGKHQPPSIIGDRWQYTGKVTEKDGITTYVYERIQSK

SEQ ID NO: 16 - protein, 67 aa, Staphylococcus lugdunensis
VPNDAPRVDIDELKITIYVDTNGREIVPSRKGQLPPEQFIGQDWQYTGHKIEKDGITTYIYKKVENA

SEQ ID NO: 17 - protein, 10 aa, Staphylococcus aureus
VVSGEKNPYV